(19) United States
(12) Patent Application Publication
(10) Pub. No.: US 2010/0174272 A1
(43) Pub. Date: Jul. 8, 2010

(54) IN-SITU REFILLABLE OPHTHALMIC IMPLANT

(76) Inventor: Alan L. Weiner, Arlington, TX (US)

Correspondence Address: ALCON, IP LEGAL, TB4-8, 6201 SOUTH FREEWAY, FORT WORTH, TX, 76134, US

(21) Appl. No.: 12/641,352

(22) Filed: Dec. 18, 2009

Related U.S. Application Data
(60) Provisional application No. 61/142,242, filed on Jan. 2, 2009.

(51) Int. Cl. A61F 9/00; A61M 37/00

(52) U.S. Cl. 604/891.1

(57) ABSTRACT

The present invention is directed to an in-situ refillable ophthalmic implant having a refill port in communication with a reservoir and a release control mechanism. The present invention also relates to methods of forming and using the ophthalmic implant. Preferably, the control release mechanism include opening[s] providing for passive passage of pharmaceutical ophthalmic composition, particularly therapeutic agent, out of the reservoir, through the opening[s] and into the eye.

### CROSS REFERENCE TO RELATED APPLICATION

This application claims priority under 35 U.S.C. §119 to U.S. Provisional Patent Application No. 61/142,242, filed Jan. 2, 2009, the entire contents of which are incorporated herein by reference.

### TECHNICAL FIELD OF THE INVENTION

The present invention is related to an in-situ refillable ophthalmic implant having a refill port and a release control mechanism. The present invention also relates to methods of forming and using the ophthalmic implant.

### BACKGROUND OF THE INVENTION

For many ocular conditions such as glaucoma, age related macular degeneration, secondary cataracts or others, it is often desirable to provide therapeutic agent to particular locations within the eye and to provide those agents over an extended time period (e.g., weeks, month or even years). Ophthalmic implants provide at least one mechanism for providing therapeutic agents in this manner. As such, the pharmaceutical industry has dedicated significant resources in the development of such implants.

U.S. Pat. No. 5,466,233 to Weiner et al. describes a tack shaped device having a post and head. The post can include a permeable membrane that forms a chamber, the chamber being filled with liquid drug that is delivered to the eye by passing through the membrane.

U.S. Pat. No. 5,707,643 to Ogura et al. describes a scleral plug having at least a portion thereof formed of a lactic acid copolymer of lactic acid units and glycolic acid units and containing a drug. The material of the plug is biodegradable for allowing drug to be released gradually over time.

U.S. Pat. No. 6,976,982 to Santini, Jr et al. describe flexible microchip devices suitable for application to the surface of an eye and designed to controllably release therapeutic agents to the eye.

While many advances have made in the arena of ophthalmic implants, there are still many drawbacks that plague conventional extended release ophthalmic implants. As one example of these drawbacks, many conventional ophthalmic implants have only a particular amount of therapeutic agent upon implantation within an eye and must be replaced once that amount of agent has been delivered. As another example of these drawbacks, many conventional ophthalmic implants lack a reliable mechanism for controlling the amount of drug released over time or the conventional implants can include overly complex mechanisms for controlling drug release. As still another example of these drawbacks, many conventional ophthalmic implants lack the ability to deliver therapeutic agents into locations substantially below the surface of the eye.

In view of the above, the present invention provides an ophthalmic implant and a method of applying and/or using the implant where the implant and/or method overcome one or more of the aforementioned drawbacks or other drawbacks commonly associated with conventional ophthalmic implants.

### SUMMARY OF THE INVENTION

The present invention is directed to an in-situ refillable ophthalmic implant.

The implant typically includes a body portion, a fill portion and a release control mechanism. The body portion defines a reservoir suitable for receipt of a pharmaceutical composition that includes a therapeutic agent. The fill portion defines a fill port in fluid communication with the reservoir for allowing the pharmaceutical composition to be repeatedly located within the reservoir. The release control mechanism includes at least one opening suitable for providing a controlled passive release of the pharmaceutical composition into the eye over an extended time period. Upon application of the implant to the eye, the release control mechanism is typically located within the eye (e.g., the vitreous of the eye) and the fill portion is located adjacent the sclera or cornea of the eye such that the fill port remains accessible outside of the vitreous of the eye and also possibly outside of the sclera, cornea or both.

The implant can include various additional or alternative components or features and can be characterized by various additional or alternative configurations. The body portion, the fill portion or a combination thereof can define a contact surface that is disposed over the sclera upon application of the implant to the eye. The at least one opening of the release control mechanism can include multiple openings wherein the multiple openings are sized to effectuate the controlled release of the therapeutic agent. The release control mechanism can include a door that can be opened and closed remotely to provide release of therapeutic agent to the vitreous. The control release mechanism can be comprised of a silicon disc through which the at least one or multiple opening[s] extend. The body portion can be overmolded onto the control release mechanism. The fill portion can include a diaphragm associated with the port, the diaphragm being penetrable by a needle or other elongated injection device for allowing filling of the is reservoir through such injection device, the diaphragm also being capable of self sealing after removal of the needle. The fill portion can include a cap portion that, upon implantation of the implant, is below the conjunctiva and resides upon the sclera.

### BRIEF DESCRIPTION OF THE DRAWINGS

FIG. 1 is a side view of an exemplary ophthalmic implant in accordance with the present invention; and

FIG. 2 is a perspective view of the exemplary implant of FIG. 1 applied to an eye of an individual.

### DETAILED DESCRIPTION OF THE INVENTION

The present invention is predicated upon the provision of an ophthalmic implant and a method of implanting and/or using that implant. The implant will typically include a body portion defining a reservoir suitable for the receipt of a pharmaceutical composition. The implant will also typically include a fill portion that will allow the implant reservoir to be initially filled with the pharmaceutical composition and will typically also allow the implant reservoir to be refilled after the implants has been implanted in an eye. The implant will also typically include a release control mechanism that can reliably control the amount of pharmaceutical composition release to the eye.

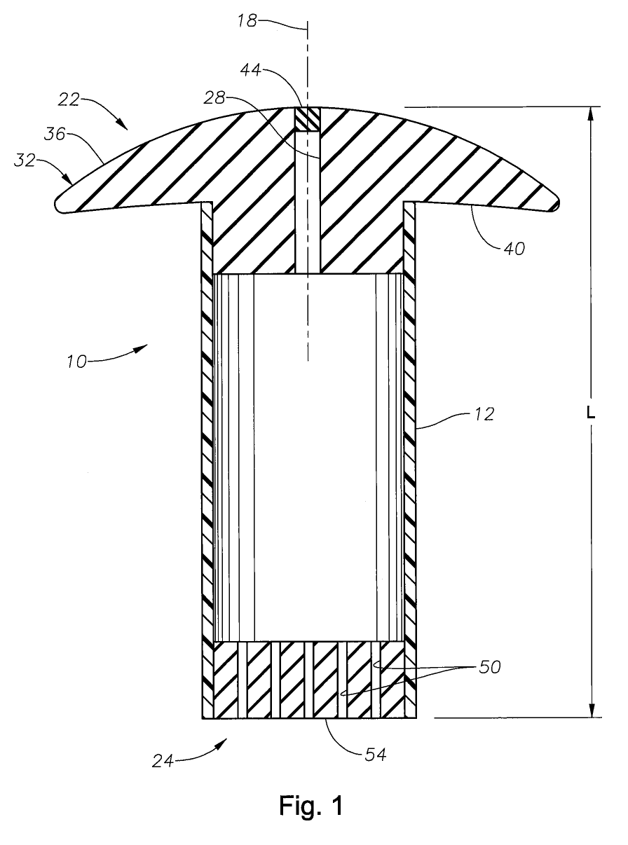

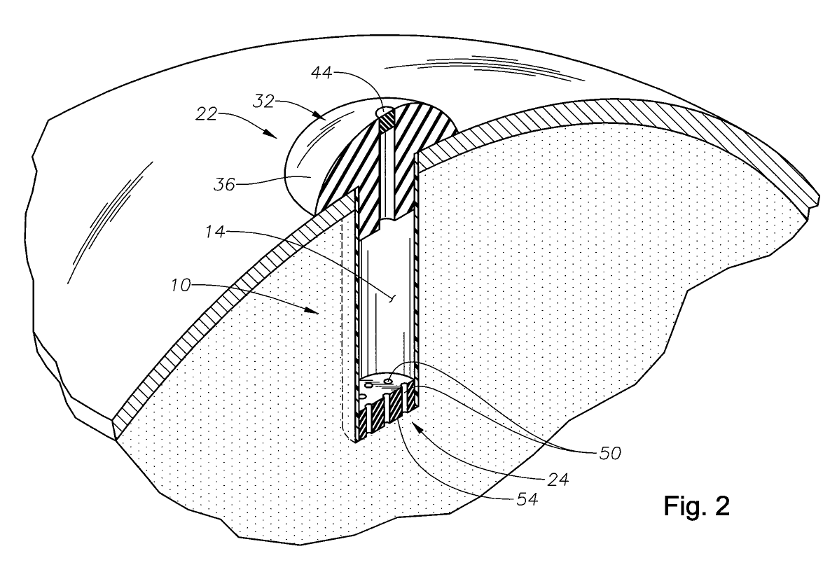

With reference to FIGS. 1 and 2, there is illustrated an exemplary in-situ s refillable ophthalmic implant 10 in accordance with the present invention. The implant 10 is illustrated as including a body portion 12, which defines a reservoir 14 within the implant 10. In the embodiment illustrated, the implant 10 is generally symmetrical about an axis 18, which extends along a length (L) of the implant 10, the body portion 12 or both.

A fill portion 22 is included at one end of the length (L) of the implant 10 and a release control mechanism 24 is included at an opposite end of the length (L) of the implant 10. The fill portion 22 is illustrated as having a port 28 suitable for aiding in the receipt of a pharmaceutical composition into the reservoir 14 of the implant 10. The fill portion 22 is also illustrated as including a cap 32 from which the body portion 12 extends.

The cap 32 may be formed integrally with and of the same material as the body portion 12. However, in the illustrated embodiment, the cap 32 is formed of a separate material from the body portion 12 and is attached to the body portion 12. The cap 32 may be attached to body portion 12 using any of a variety of fastening mechanisms, but preferably involves an interference fit with a portion of the cap 32 extending partially into the body 12 or a portion of the cap 32 extending about the body 12 externally.

In a preferred embodiment, the cap 32 is formed of a relatively soft material (e.g., a polymeric material) that is biocompatible with the human eye. Examples of preferred materials include, without limitation, silicone, parylene, an acrylic material or the like. In the illustrated embodiment, the port 28 extends centrally through the cap 32 and the cap 32 is annular about the port 28.

The cap 32 of the fill portion 22 includes an external surface 36 that is designed to be external of and face outwardly away from the eyeball including the vitreous of the eye, the sclera of the eye or both after the implant 10 is surgically applied to the eye. The external surface 36 is illustrated as being generally convex. Advantageously, when used, the convex surface and material of the cap 32 can aid in allowing for the implant 10 to reside in its intended location within the eye without causing significant irritation or discomfort.

The cap 32 of the fill portion 22 is also shown to include a contacting surface 40 that is designed to contact the sclera or conjunctiva after the implant 10 has been applied to the eye. In a preferred embodiment, the contacting surface 40 can be slight convex for better accommodation of the sclera or conjunctiva. In one particular embodiment, the cap 32, the fill portion 22 or both are disposed within or over the pars plana of the eye over or near the limbus.

An access element 44 will typically be associated with the port 28 for selectively restricting movement of fluid through the port 28. The access element 44 can be a removable plug, a door, a valve or other such element. In one preferred embodiment, the access element 44 is a diaphragm, which can be opened through is penetration by a needle or other delivery device but will also close to again restrict fluid flow after removal of the needle or other delivery device from the diaphragm. In such an embodiment, it is contemplated that the cap 32 and the access element 44 (i.e., the diaphragm) could be integrally formed as a singular part of the same material. In such an embodiment, silicone (e.g., a non-coring silicone), parylene or another material could ideally be used and a thin portion of the cap 32 that acts as a diaphragm 44. Advantageously, a needle or other device can be extended through these materials and any opening made by the needle will typically self close and/or seal after removal of the needle or other device.

The body portion 12 is illustrated as being annular, and more particularly cylindrical, for defining the reservoir 14. The body portion 12 may be formed of a variety of materials (e.g., polymer or metal materials) that are biologically compatible with the human eye. Exemplary suitable materials include, without limitation, parylene, polyetheretherketone (PEEK), polyethylene, polyimide, ethylene vinyl acetate, acrylic polymers, combinations thereof or the like.

The control release mechanism 24 will typically include one or more opening[s] 50 through which material (e.g., fluid that contains therapeutic agent) can pass. The use of multiple openings 50 is generally preferable and there is typically at least 3, more typically at least 6 and even more typically at least 10 openings and there is typically no greater than 1000, more typically no greater than 200 and even more typically no greater than 50 openings.

It is preferable that the control release mechanism 24 may be configured for passive passage of material through the opening[s] 50. Thus, flow through the opening[s] 50 is generated or driven through natural diffusion and/or equilibrium mechanisms. The control release mechanism may consist or consist essentially of the opening[s] 50 and the material through which the openings extend. Alternatively, the control release mechanism 24 can include mechanical mechanisms for selectively inhibiting or allowing the passive passage of material through the openings 50. Examples of such mechanism include valves or doors, which can be selectively and even remotely (e.g., through radio frequency signaling) opened and closed to respectively allow and inhibit passage of material through the opening[s] 50. As used herein, the terms opened and closed as they refer to the control release mechanism include partial and full opening or close. Moreover, it is contemplated that partial opening or closing of the mechanism may be employed to further control the amount of diffusion or movement of fluid through the opening[s] 50 thereby further controlling the deliver of the pharmaceutical composition to the eye.

Material, particularly ophthalmic pharmaceutical composition and aqueous humor fluid, is typically allowed to freely flow and/or diffuse into and out of the reservoir 14 with the size of the opening[s] 50 assisting in controlling the rate of flow and/or diffusion into and out of the reservoir 14. The opening[s] 50, particularly for a passive system, have a cross-sectional area that controls the rate at which material, particularly therapeutic agent, flows out of the reservoir and into the eye. That cross-sectional area is typically at least 8 microns², more typically at least 15 microns² and even more typically at least 50 microns². That same cross-sectional area is also typically no greater than 4000 microns², more typically no greater than 2000 microns² and still more typically no greater than 500 microns². The cross-sectional area of the opening, as used herein, is any sectional area of the opening wherein the outer perimeter of the opening is fully defined by the material of the control release mechanism and wherein, for fluid to pass through the opening into or out of the reservoir 14, it must also pass through the cross-sectional area.

In the illustrated embodiment, the control release mechanism 24 is a plate 54 through which the opening[s] 50 extend. The plate 54 has opposing substantially parallel surfaces through with the opening[s] 50 extend. In the embodiment shown, the opening[s] 50 or cylindrical in shape although they may be shaped otherwise as well. The opening[s] 50 typically have a diameter of at least about 0.2 microns, more typically at least about 2 microns and even more typically at least about 8 microns. The diameter of the opening[s] illustrated is also typically no greater than about 100 microns, even more typically no greater than 40 microns and even more typically no greater than about 25 microns. While it is understood that a generally uniform distribution of the opening[s] 50 over the surface of the plate 54 is desirable other non-uniform distribution of opening[s] 50 are also possible. A suitable thickness for the plate will typically be at least about 0.05 mm, more typically at least about 0.08 mm and will typically no greater than 0.5 mm and more typically no greater than 0.3 mm.

In the illustrated embodiment, the length (L) of the implant 10 will typically be less than about 15 mm, more typically less than 10 mm and even more typically less than 8 mm. Also in the illustrated embodiment, the outer diameter of the body is portion 12 of the implant 10 will typically be less than 7 mm more typically less than 4 mm and even more typically less than 2.5 mm. The length of the implant is typically sufficiently small such that it does not interfere with the vision or field of view of the eye.

The control release mechanism 24, and particularly the plate 54, may be formed of a variety of materials such as metals or polymeric materials. In a preferred embodiment, however, it is formed of an etchable material such as silicon, which allows the opening[s] 50 to be etched into the material.

The control release mechanism 24, and particularly the plate 54, can be attached to the body portion 12 of the implant 10 using an interference fit or other fastening technique. In one preferred embodiment, the body portion 12 is overmolded onto the plate 54 for attaching the plate 54 to the body portion 12. Other suitable fastening techniques could involve the use of sealing members, adhesive, fasteners, specially designed attachment members or the like. It is further contemplated that the body portion 12 and the control release mechanism 24 could be integrally formed of the same material.

For implantation, the implant 10 is typically inserted into a surgical incision in the eye. Once implanted, the implant 10 may be held in place with sutures or other mechanisms. Additionally or alternatively, it is contemplated that the body portion 12 or other portion of the implant 10 may be shaped to assist in maintaining the implant 10 in place within the eye. As one example, the body portion 12 may have a spiral configuration such that the body portion 12 itself substantially maintains the implant 10 in place in the eye. An example of such a spiral configuration is illustrated in U.S. Pat. No. 6,719,750 to Varner et al, which is fully incorporated herein by reference for all purposes.

Generally, the implant 10 may be located in a variety of locations within the eye. In one preferred embodiment, the implant 10 is surgically positioned such that the body portion 12 extends into the vitreous of the eye and the fill portion 22, particularly the cap, is located between the conjunctiva of the eye and the vitreous of the eye. In a highly preferred embodiment, the cap 22 is beneath the conjunctiva of the eye, the surface 40 of the cap 22 contacts the sclera of the eye and the body portion 12 extends through the sclera into the eye.

The pharmaceutical composition that is provided within the implant 10 will typically include a therapeutic agent and that agent may or may not be provided within a pharmaceutical vehicle. The therapeutic agent of the present invention may be provided in various forms within the implant and, when used, could be provided with various different pharmaceutical vehicles (e.g., water alone or combined with additional ingredients). The agent could be a solid, semi-solid or liquid within the implant. As one example, the therapeutic agent could be provided as a solid within a liquid (e.g., aqueous) suspension. As another example, the therapeutic agent could be provided as an oil without any vehicle at all.

It is generally preferable that the pharmaceutical composition be injectable with a syringe. Thus, it is preferable that the pharmaceutical composition be liquid or semi-solid even when the therapeutic agent may be entirely or substantially entirely solid (e.g., a suspended solid). Such liquid or semi-solid compositions can be injected into the implant 10 with a syringe prior to insertion of the implant 10 within an eye and/or after insertion of the implant 10 within an eye. Thus, the implant 10 may be filled and then re-filled one or multiple times.

Non-limiting examples of potential ophthalmic therapeutic agents for the present invention include: anti-glaucoma agents, anti-angiogenesis agents; anti-infective agents; anti-inflammatory agents; growth factors; immunosuppressant agents; and anti-allergic agents. Anti-glaucoma agents include beta-blockers, such as betaxolol and levobetaxolol; carbonic anhydrase inhibitors, such as brinzolamide and dorzolamide; prostaglandins, such as travoprost, bimatoprost, and latanoprost; seretonergics; muscarinics; dopaminergic agonists. Anti-angiogenesis agents include anecortave acetate (RETAANE™, Alcon™ Laboratories, Inc. of Fort Worth, Tex.) and receptor tyrosine kinase inhibitors (RTKi). Anti-inflammatory agents include non-steroidal and steroidal anti-inflammatory agents, such as triamcinolone actinide, suprofen, diclofenac, ketorolac, nepafenac, rimexolone, and tetrahydrocortisol. Growth factors include EGF or VEGF. Anti-allergic agents include olopatadine and epinastine. The ophthalmic drug may be present in the form of a pharmaceutically acceptable salt.

Advantageously, the opening[s] 50 of the implant 10 can act as a simple mechanism for controlling the release of the pharmaceutical composition, particularly the therapeutic agent, over time. In a preferred embodiment, the implant 10 includes opening[s] 50 sized to include the cross-sectional areas discussed above. In such an embodiment, the opening[s] 50 can operate to release at least 50%, more typically at least 80% and even more typically at least 90% of an amount of therapeutic agent located within the implant 10 over a period of time that is at least 48 hours, more typically at least 7 days and even more typically at least 60 days but is no greater than 5 years, more typically no greater than one year and still more typically no greater than 6 months.

The initial amount of pharmaceutical composition including therapeutic agent can be disposed within the reservoir 14 during assembly of the implant 10 or thereafter. For refilling the implant 10, a device such as a syringe is used to extend a needle through the access element 44, the port 28 or both and push pharmaceutical composition into the reservoir 14. For aiding in refill, it may desirable to use one device (e.g., syringe) to aspirate material (e.g., aqueous humor liquid) from the reservoir 14 and another device (e.g., syringe) to push pharmaceutical composition into the reservoir 14 thereafter. Alternatively, a single syringe device can be created to concurrently aspirate fluid from the reservoir 14 while replacing that fluid with pharmaceutical ophthalmic composition.

The entire contents of all cited references are specifically incorporated by reference into this disclosure for all purposes. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

We claim:

1. An in-situ refillable ophthalmic implant, comprising:
a body portion defining a reservoir;
a fill portion defining a fill port that is in fluid communication with the reservoir for allowing a pharmaceutical composition to be repeatedly located within the reservoir, the pharmaceutical composition including a therapeutic agent; and
a release control mechanism having at least one opening suitable for providing a controlled passive release of the pharmaceutical composition into the eye over an extended time period;
wherein, upon application of the implant to the eye, the release control mechanism is located within the eye and the fill portion is located outside the vitreous of the eye adjacent the sclera of the eye such that the fill port remains accessible outside of the vitreous of the eye.

2. An implant as in claim 1 wherein the body portion, the fill portion or a combination thereof define a contact surface that is disposed over the sclera upon application of the implant to the eye.

3. An implant as in claim 1 wherein the at least one opening of the release control mechanism includes multiple openings and wherein the multiple openings are sized to effectuate the controlled release of the therapeutic agent.

4. An implant as in claim 1 wherein the release control mechanism includes a door that can be opened and closed remotely to provide release of therapeutic agent to the vitreous.

5. An implant as in claim 1 wherein the body portion is elongated with a first end opposite a second end, the fill portion being located at the first end and the release control mechanism being located at the second end.

6. An implant as in claim 1 wherein the therapeutic agent lowers intraocular pressure within the eye.

7. An implant as in claim 1 wherein the control release mechanism includes a silicon disc through which the at least one opening extends.

8. An implant as in claim 1 wherein the body portion in overmolded onto the control release mechanism.

9. An implant as in claim 1 wherein the body portion, the fill portion or both are formed of a polymeric material.

10. An implant as in claim 1 further comprising a removable plug located within the port.

11. An implant as in claim 1 further comprising a diaphragm associated with the port, the diaphragm being penetrable by a needle or other elongated injection device for allowing filling of the reservoir through such injection device.

12. An implant as in claim 1 wherein, upon implantation, a cap portion of the implant is below the conjunctiva and resides upon the sclera.

13. An implant as in claim 1 wherein, upon implantation, the release control mechanism is located within the vitreous of the eye.

14. An implant as in claim 1 wherein the at least one opening has a cross-sectional area that is at least 8 microns² but is no greater than 4000 microns².

15. An implant as in claim 1 wherein the at least one opening has a cross-sectional area that is at least 15 microns² but is no greater than 2000 microns².

16. An in-situ refillable ophthalmic implant, comprising:
a body portion defining a reservoir;
a fill portion defining a fill port that is in fluid communication with the reservoir for allowing a pharmaceutical composition to be repeatedly located within the reservoir, the pharmaceutical composition including a therapeutic agent; and
a release control mechanism having multiple openings suitable for providing a controlled passive release of the pharmaceutical composition into the eye over an extended time period;
wherein, upon application of the implant to the eye, the release control mechanism is located within the vitreous of the eye and the fill portion is located outside the vitreous of the eye adjacent the sclera of the eye such that the fill port remains accessible outside of the vitreous of the eye; and
wherein the body portion, the fill portion or a combination thereof define a contact surface that is disposed over the sclera upon application of the implant to the eye; and
wherein the control release mechanism includes a silicon disc through which the multiple openings extend; and
wherein each of the multiple openings has a cross-sectional area that is at least 8 microns² but is no greater than 4000 microns².

17. An implant as in claim 16 wherein each of the multiple openings has a cross-sectional area that is at least 15 microns² but is no greater than 2000 microns².

18. An implant as in claim 17 wherein the body portion, the fill portion or both are formed of a polymeric material.

19. An implant as in claim 18 further comprising a diaphragm associated with the port, the diaphragm being penetrable by a needle or other elongated injection device for allowing filling of the reservoir through such injection device.

20. An implant as in claim 19 wherein the body portion is elongated with a first end opposite a second end, the fill portion being located at the first end and the release control mechanism being located at the second end.
